(19) United States
(12) Patent Application Publication
(10) Pub. No.: US 2022/0040393 A1
(43) Pub. Date: Feb. 10, 2022

(54) Device and Method for Attenuation of CO2 in Circulating Blood

(71) Applicant: RESPIROGEN, INC., Boulder, CO (US)

(72) Inventor: Robert M. SCRIBNER, Longmont, CO (US)

(21) Appl. No.: 17/393,293

(22) Filed: Aug. 3, 2021

Related U.S. Application Data
(60) Provisional application No. 63/061,023, filed on Aug. 4, 2020.

(51) Int. Cl. A61M 1/36; A61M 1/32; A61M 1/28

(52) U.S. Cl. CPC A61M 1/3679; A61M 1/32; A61M 2202/0225; A61M 2210/1064; A61M 2202/0208; A61M 1/28

(57) ABSTRACT

Systems and methods for the removal of carbon dioxide from circulating blood via the use of microbubbles within a body cavity, optionally including the simultaneous provision of oxygen to the circulatory system. Through placement of microbubbles within a body cavity and the use of a carbon dioxide scavenging catheter, carbon dioxide and/or oxygen exchange may occur. Overall improvement in extending survival rate time during emergency situations caused by pulmonary or similar ventilation restricting injury and/or failure may be achieved.

### CROSS REFERENCE TO RELATED APPLICATIONS

This application claims the benefit of U.S. provisional application 63/061,023 entitled “Device and Method for Attenuation of CO2 in Circulating Blood,” filed Aug. 4, 2020, the disclosure of which is incorporated by reference herein in its entirety.

### FIELD OF THE INVENTION

The invention relates generally to ventilating a subject via artificial means. More particularly, the invention relates to devices, system and methods of removing waste products such as carbon dioxide from and/or delivering oxygen to a subject via a body cavity using microbubble carriers.

### BACKGROUND OF THE INVENTION

Oxygen is one of the basic essentials for sustaining life. Today's medical technology can supply oxygen to patients experiencing pulmonary failure, otherwise known as respiratory failure. Pulmonary failure occurs when the lungs experience significant damage and are unable to supply the body and brain with oxygen. Pulmonary failure may be caused by a variety of conditions including, for example, lung cancer, physical trauma, acute respiratory distress syndrome (ARDS), COVID, aerosolized bioterrorism agents, and diseases such as severe acute respiratory syndrome (SARS), pneumonia, tuberculosis, sepsis, and other bacterial or viral infections, physical trauma, and chemical or smoke inhalation. Currently, oxygen can be supplied to patients experiencing pulmonary failure through mechanical ventilation (MV) or extracorporeal membrane oxygenation (ECMO). However, the mortality rate of patients receiving oxygen through MV or ECMO remains high.

MV has been an ineffective method for delivering oxygen to the body in certain cases because oxygen exchange is decreased by damage to the lung and because of increased stress caused to the injured lung by the treatment. As an example, for patients experiencing severe hypoxemia arising from lung injury MV may be inadequate owing to limited mass transfer in the injured lung; over-inflation, barotrauma and cyclic closing and reopening of the alveoli may further damage the lung and trigger a pulmonary and systemic inflammatory reaction that may lead to multiple system organ failure.

ECMO is a temporary artificial extracorporeal support of the respiratory system and/or cardiac system. ECMO was first used in an adult in 1972 to treat severe respiratory failure and in 1974 on a first newborn. Innovations in ECMO include the introduction of polymethylpentene hollow fibers with non-thrombogenic coatings and thin wire-reinforced cannula walls. ECMO use has historically centered on neonatal care. However, ECMO is an expensive alternative therapy with limited availability in hospitals and length of treatment. ECMO is able to bypass the injured lungs to deliver oxygen and allow the lungs to heal, however there is a high risk of thrombosis and contamination of the blood because it is removed from the body. Since ECMO is associated with significant complications, sometimes additional therapies are required such as the use of anticoagulants (heparin is standard). However, anticoagulants are often administered to the patient leading to additional adverse side effects. Additionally, ECMO is expensive and complex to operate, limiting its accessibility for emergency care.

Because of the high mortality rate, methods of bypassing the lungs and delivering oxygen directly to the body have been explored for many years. Research has focused on peritoneal oxygenation as a method of extrapulmonary respiration. The exchange of oxygen and carbon dioxide occurs through the large surface area of the membrane that lines the abdominal cavity, the peritoneum.

Previous methods include in situ extrapulmonary ventilation (EV). Limited success in oxygenating blood in situ has been achieved by circulating fluorocarbons, blood, and liposome-encapsulated hemoglobin (a synthetic oxygen carrier) through the intraperitoneal (IP) space, or cavity. Additional previous methods include the study of carriers for the delivery of oxygen. Carriers have included blood, perfluorocarbon (PFC), and synthetic hemoglobin carriers, for example TRM-645, which are effective. In contrast, pure oxygen gas has been shown not to be an effective medium for increasing blood oxygen saturation via the peritoneum. None of the effective carriers, however, are both safe and economical. For example, PFCs are expensive to generate and evaporate into potent greenhouse gases creating a significant environmental concern. They are also very stable, tending to accumulate in biological systems in which they are used. Blood and products derived from blood (like synthetic hemoglobin carriers) suffer from scarcity and are relatively expensive to fabricate and store. Furthermore, EV ventilation requires high volumes of perfusate; therefore, a fluid that is economical and biodegradable is important. In additional, none of the previously developed methods have achieved certain components for extrapulmonary respiration: (1) delivery of an adequate supply of oxygen, (2) long-term safety for the patient, and (3) cost-effectiveness.

Another form of therapy explored to deliver oxygen systematically by circulating it through the IP space is a method that uses an oxygen microbubble (OMB) carrier. OMBs are oxygen filled bubbles that have a shell composed of a phospholipid monolayer. The phospholipid monolayer shell of an OMB has similar composition to lung surfactant and requires comparable physical properties, such as rapid adsorption to and mechanical stabilization of the gas/liquid interface and high gas permeability. Thus OMBs are also designed to mimic the mechanical and gas transport properties of the alveolus to deliver the oxygen payload and uptake carbon dioxide.

Previous research has focused on the delivery of OMBs through intravenous (IV) oxygen delivery. However, IV injection of OMBs is a one-way administration and does not allow for the circulation of microbubbles into and out of the body to both deliver oxygen and remove carbon dioxide. Delivering oxygen using an IV may be viable for short-term increase in oxygen saturation, but the prolonged continuous infusion of oxygen microbubbles into the bloodstream poses significant challenges for clinical translation, including the potential for embolism, thrombosis, immunogenicity and toxicities of lipid and saline load. For example, with IV injection of OMBs, any oxygen inspired through the lungs can be absorbed by the microbubbles and can cause long-circulating bubbles that may cause embolism or other problematic conditions, such as those observed in decompression sickness. Further, with the potential for embolism, IV injection of microbubbles requires a strict upper limit on the microbubble size (<10 micrometers) and on the microbubble volume fraction (<70%).

Still another problem with IV injection of OMBs is that any nitrogen inspired through the lungs, such as that found in air, will be absorbed by the microbubbles. Thus, the microbubbles will exchange oxygen for nitrogen. The nitrogen-containing microbubbles will be persistent, which can lead to serious problems, such as those observed during decompression sickness and embolism (thus leading to severe morbidity and death). In addition, intravenous oxygenation using OMBs cannot support long-term ventilation due to the lipid and saline load from continuous infusion. Moreover, allowing microbubbles to remain within the circulatory system allows any waste materials potentially absorbed by the microbubbles to remain there as well.

A demand therefore exists for a system and method for delivery of oxygen and removal of wastes from a subject that is more effective and efficient than the current systems and methods presently available. The invention satisfies this demand.

### SUMMARY OF THE INVENTION

The invention is a system and methods that desirably allows for the provision of oxygen to and/or removal of carbon dioxide from the circulatory system of a patient using microbubbles, including oxygen microbubbles (OMBs) or other microbubbles present in a body cavity. Although the invention is discussed in reference to the intraperitorneal (IP) cavity, the invention is also applicable to other cavities of the subject such as the gastrointestinal (GI), pleural, cranial, vertebral, pericardial and other cavities as more fully described below. The invention desirably includes the delivery of microbubbles or OMBs to a body cavity, in combination with the introduction of a device capable of providing supplemental oxygen and/or carbon dioxide removal that is in contact with the OMBs within the body cavity, wherein the actions of such device can dramatically extend the amount of time that the OMBs are able to provide safe and adequate patient ventilation and/or waste removal for the patient.

According to the invention, microbubbles can be designed for high oxygen carrying capacity, high oxygen delivery rate and sufficient stability for storage, transport and usage. Moreover, microbubbles can allow and/or drive the transport of gases through and/or between the wall structures of adjacent bubbles, which can allow for the diffusion of gases from a region of higher concentration to a region of lower concentration, which may even occur in opposing directions for differing gases. In the present invention, microbubbles containing one or more gases may be introduced into a body cavity, and a source of oxygen and/or scavenger or carbon dioxide may be introduced into the body cavity, wherein a higher gradient of oxygen within the oxygen source and/or within the microbubbles themselves (such as in the case of the initial introduction of oxygen microbubbles) can induce a diffusive or other flow of oxygen through the tissue walls of the body cavity for uptake into the patient's circulatory system. Alternatively or concurrently, the carbon dioxide scavenger can create a region of lower carbon dioxide concentration within the body cavity, which can induce a diffusive or other flow of carbon dioxide out of the patient's circulatory system, through the tissue walls of the body cavity, into and through the microbubbles and into the CO₂ scavenging unit. In this manner, direct systemic oxygenation and carbon dioxide removal (or CDR) using microbubbles within a peritoneal or other space is a radical change from existing oxygen delivery platforms.

In some embodiments, the microbubbles utilized herein could be circulated through a body cavity such as the peritoneal cavity. Circulating OMBs through a body cavity would desirably require less equipment and technical expertise than initiating and maintaining an ECMO circuit. Thus, body cavity ventilation using OMBs may provide extrapulmonary ventilation (EV) therapy to medical facilities that presently lack the funding or highly trained personnel required for operating an ECMO system.

By allowing OMB's or other microbubbles to reside within a body cavity, optionally with circulating of OMBs in the body cavity, oxygen and carbon dioxide exchange desirably occurs across the peritoneum, the membrane of the abdominal cavity. Additionally, unlike IV injection of microbubbles which cannot remove carbon dioxide, a body cavity injection using OMBs according to the invention can beneficially remove carbon dioxide. Additionally, the IP injection of OMBs, for example, is not subject to a strict upper limit of the microbubble size and volume fraction since the microbubbles are injected into the IP space (not the vasculature) and can thus easily be removed. This provides oxygen to a subject, for example after it experiences a right pneumothorax. This significantly extends life in a subject with an acute lung injury hypoxemia or similar situation in which the lung function is sub-optimal.

Direct systemic ventilation using OMBs within a body cavity space is a radical change from existing oxygen delivery platforms. For example, the invention may be used to supplement ventilation in cases of airway failure, lung injury, respiratory distress, cardiac arrest, or other situations in which lung function is sub-optimal. The invention also precludes the need for an extracorporeal loop, thus potentially circumventing the complications of thrombosis and immune reaction presented by the artificial surfaces exposed by ECMO. The prospect of providing a bridge to, or even supplanting ECMO, may significantly reduce the cost and complexity of EV.

The ability to deliver oxygen via a body cavity may also have significant clinical implications. For example, acute severe hypoxia of any origin (due to airway obstruction or due to other causes) results in irreversible brain injury within minutes. Administration of readily accessible oxygen-bearing microbubbles may prevent the morbidity and mortality associated with acute hypoxia in many subjects such as human subjects. In addition, subjects suffering from lung injury, which represent a significant percentage of those requiring intensive care, may benefit from the delivery of oxygen that offers minimally invasive extrapulmonary oxygen supplementation. Thus, ventilator-induced lung injury may be minimized while avoiding critical hypoxia. Increasing systemic oxygen saturations may improve hypoxic pulmonary vasoconstriction and reduce pulmonary vascular resistance in subjects with acute exacerbations of pulmonary hypertension. Infants, for example, born with cyanotic congenital heart disease, could benefit from an effective delivery of oxygen that may lessen their hypoxemia preoperatively, as well as during the early postoperative recovery period. This therapy could also provide care for cases of irreversible pulmonary failure and, hence, act as a bridge to lung transplant. This work may be translated to a medical therapeutic device with the aim of replacing or augmenting ECMO with a less-invasive method of ventilation.

In addition to and/or in place of oxygen delivery, microbubbles can similarly be utilized to removed carbon dioxide and/or other substances from the circulatory system of a patient. In many instances, patient injury and/or death can be attributed primarily to an unwanted buildup of carbon dioxide within a patient's body, leading to carbon dioxide toxicity, hypercapnia and/or respiratory acidosis. —even where sufficient oxygen to maintain vital functions may be present. By removing carbon dioxide from a patient's circulatory system and preventing such toxicity, the present inventions can more adequately mimic natural ventilation, thereby improving outcomes for a variety of treated patients.

According to the invention, microbubbles and/or OMBs can be injected into a body cavity along with placement of a supply/scavenger system and allowed to deliver oxygen to and/or absorb carbon dioxide from surrounding body cavity tissues. If necessary, the microbubbles maybe periodically refreshed and/or flushed, including optionally using a saline flush to remove them from the cavity, followed by another injection of OMBs into the cavity. This process cycle can be repeated if or as often as necessary. In the alternative, OMBs may be continuously circulated through a cavity to release oxygen and absorb carbon dioxide and other gases.

Since blood is oxygenated by OMBs administered through the body cavity, along with commensurate carbon dioxide removal, the possible infusion of OMBs directly into circulation is avoided. In addition, the upper size limit (about 10 μm diameter) is not required to avoid vascular occlusion. In certain embodiments, larger microbubbles (about 10-25 μm diameter) can be perfused through the body cavity without fear of occlusion because they are separated by gas-permeable membranous tissue from the intravascular space. Thus, the effects of larger microbubble size distributions on microbubble suspension viscosity and oxygen-release rate (at equivalent volume fraction) may be measured. According to the invention, microbubbles may be between 1-25 μm in diameter with larger microbubbles being about 9-21 μm in diameter and smaller microbubbles being about 1-8 μm in diameter. However, it is contemplated that microbubbles may be between 1-100 μm in diameter and even between 1-500 μm in diameter. In addition, mixtures of microbubbles may comprise microbubbles of different sizes. The sizes of the OMBs contained within any one mixture may be only smaller microbubbles, only larger microbubbles or a combination of both smaller and larger microbubbles.

In embodiments in which a perfusate is administered, the perfusate may be about 60%, 65%, 70%, 75%, 80%, 85%, 90%, or 95%. In certain other example embodiments, the OMB perfusate may be between 10%-90%.

One advantage of the invention over previously developed methods for extrapulmonary respiration is that the invention delivers an adequate supply of oxygen, removes carbon dioxide, provides long-term safety for the subject, and is cost-effective.

One advantage of the invention is that problems associated with ECMO are addressed and have the potential to significantly increase survivability of reversible pulmonary failure caused by pathologies and dysfunctions such as heart disease, chronic lower respiratory disease, accidents, influenza and pneumonia, airway failure, lung injury, respiratory distress syndrome, and other causes. In addition, the invention may help subjects survive avian flu, severe acute respiratory syndrome (SARS), and COVID.

Other advantages of the invention include: reduced thrombogenic and immunogenic effects owing to lack of direct contact with blood, reduced risk of embolism by the OMBs or their lipid remnants because they are not in circulation, and/or prevention of blood acidosis due to excessive levels of carbon dioxide dissolved in the blood.

An additional advantage of the invention is stability of the OMBs. OMBs are stable at high volume fractions (>90%) for several weeks in refrigerated storage.

Another advantage is that OMBs administered intravenously (<70 vol %) possess a rapid dissolving property and release of oxygen in the presence of oxygen-deficient media in vitro.

Additionally, OMBs according to the invention assist in further sustaining life during 15 minutes of complete tracheal occlusion in vivo. Thus, OMBs are a promising platform for non-extracorporeal oxygen delivery.

These and other features, aspects, and advantages of the invention will become better understood with regard to the following description, appended claims, and accompanying drawings.

### BRIEF DESCRIPTION OF THE DRAWINGS

FIG. 1 illustrates an example embodiment of the ventilation system according to the invention;

FIG. 2 illustrates an example embodiment of a supply device according to the invention;

FIG. 3 illustrates one embodiment of a ventilation system used in the delivery of oxygen to a subject according to the invention;

FIG. 4 illustrates another embodiment of a ventilation system used in the delivery of oxygen to a subject according to the invention;

FIG. 5 illustrates an exemplary computer system according to the invention;

FIG. 6 illustrates an embodiment of the control scheme to monitor the perfusion of OMBs during delivery of oxygen to a subject according to the invention;

FIG. 7 illustrates a graph of survival rates over time for test subjects with a pneumothorax model and intraperitoneal injection of oxygen microbubbles compared to saline injection according to the invention;

FIG. 8 illustrates a graph of survival rates over time for test subjects with an asphyxiation model and pleural injection of oxygen microbubbles compared to saline injection according to the invention; and

FIG. 9 illustrates one exemplary embodiment of a CO₂ removal catheter for use with various embodiments of the disclosed system.

### DETAILED DESCRIPTION OF EXAMPLE EMBODIMENTS

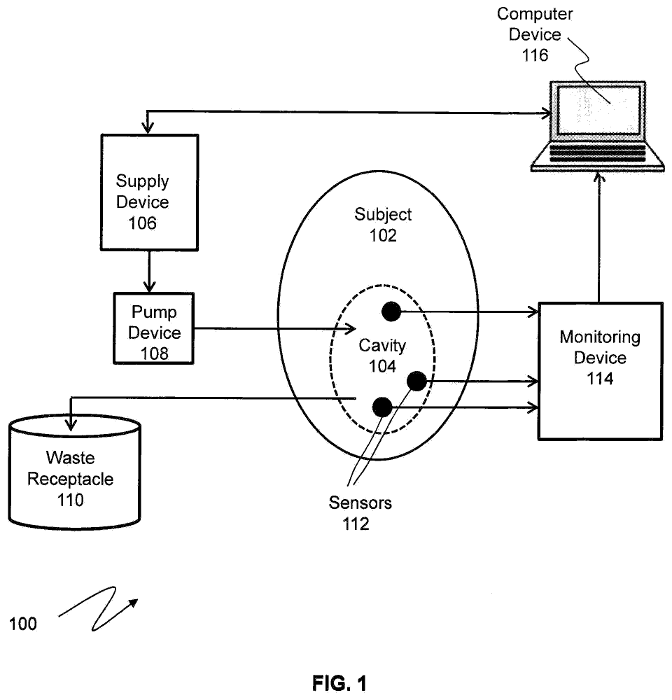

FIG. 1 illustrates an example embodiment of the ventilation system 100. In this example embodiment, a subject 102 is treated with oxygen microbubbles (OMB) dispensed from a supply device 106 through the subject's body cavity 104 by a pump device 108. As OMBs from the supply device 106 are pumped into the body cavity 104 of the subject 102, waste is simultaneously removed from the subject and dispensed to the waste receptacle 110. The monitoring of several life sustaining data is performed with sensors 112 connected to the subject 102 and the monitoring device 114. Data include, for example, intra-abdominal pressure. The intra-abdominal pressure is measured to avoid high internal pressures that could damage the body's internal organs. Other data that may also be monitored include blood gas levels of oxygen and carbon dioxide, heart pulse, blood pressure, heart ejection fraction and similar cardiac data. The monitoring device 114 transmits the data to a computer device 116 where the data is processed, for example, tabulated into a database, analyzed, and/or used to provide feedback to the supply device 106, for example to adjust the amount of OMB delivered to the subject 102. It is also contemplated that the supply device 106 transmits information to the computer device 116 for processing, for example, to adjust the data the sensors 112 monitor.

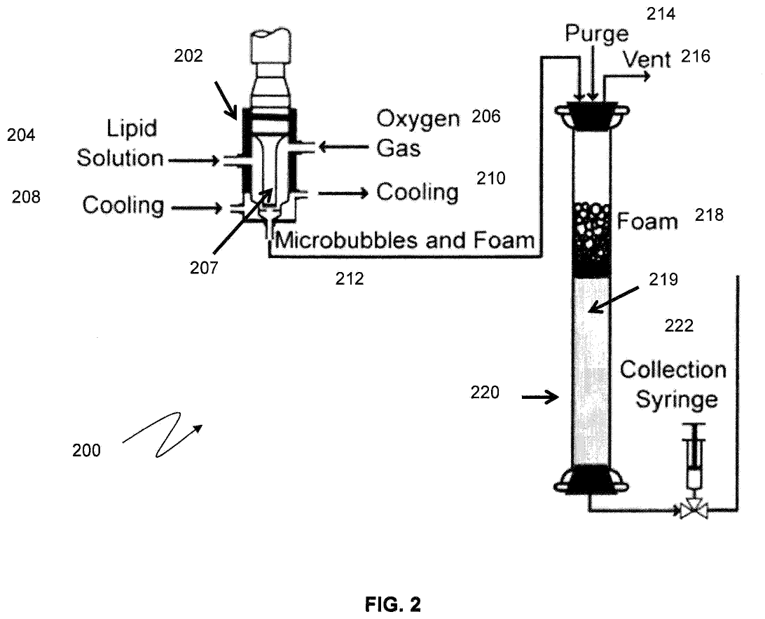

FIG. 2 illustrates an example embodiment of a supply device 200 according to the invention. The supply device 200 includes a reaction chamber 202 with inlets for a lipid solution 204, and oxygen gas 206. The lipid solution 204 and oxygen gas 206 are mixed to create a mixture 207, which is then cooled. The mixture 207 is cooled using material such as air or liquid passing through an inlet 208 and outlet 210. In this process, a mix 212 of microbubbles and foam is created. The microbubbles and foam mix 212 is passed to a collection container 220. A solution 214, such as saline, is purged into the collection container 220 to combine with the microbubbles and foam mix 212. The collection container 220 includes a vent 216 for the discharge of gas. As the microbubbles and foam mix 212 combine with solution 214, ambient gas is vented 216 and the foam 218 separates from the microbubbles 219. The microbubbles 219 are then collected in a syringe 222, centrifuged and stored for future use.

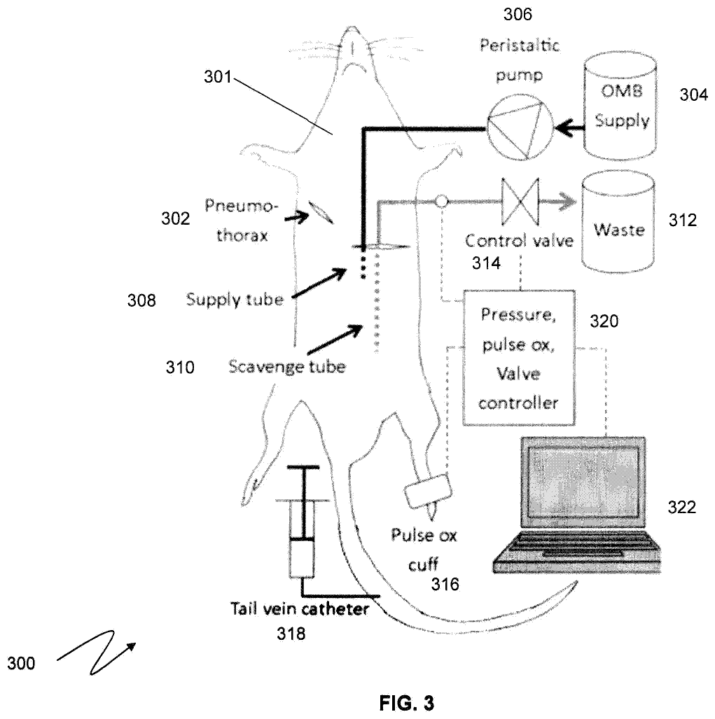

FIG. 3 illustrates one embodiment of a ventilation system 300 used in the delivery of oxygen to a subject according to the invention. In this example embodiment, a subject 301 has a right pneumothorax 302. Cardiac arrest eventually follows a right pneumothorax and is caused by lack of blood oxygen (hypoxemia) and by elevated carbon dioxide levels and subsequent low blood pH (acidosis) that result from pulmonary failure. To keep the subject alive longer while life-saving treatment can be administered, the ventilation system 300 according to the invention is employed. A small incision is made into the abdomen of the subject 301 to give access to the IP cavity. Oxygen microbubbles from the supply device 304 are circulated into the IP cavity though a supply tube 308 with a peristaltic pump 306. Additionally a scavenge tube 310 is used to remove fluid waste 312, which may be controlled by a valve 314.

The effectiveness of IP extrapulmonary respiration is established by measuring and monitoring several life sustaining data. Intra-abdominal pressure may be measured at the supply tube 308 inlet. This is measured to avoid high internal pressures that could damage the body's internal organs. The blood gas levels of oxygen and carbon dioxide, heart pulse, blood pressure, heart ejection fraction. Similar cardiac data may be measured with a paw cuff pulse 316 or similar medical device and perform periodic blood draws. Additionally drug dosage in the blood may be measured through a catheter 318. Measuring and monitoring these and other life sustaining data may be performed with a monitoring device 320 and viewed on a monitor of a computer device 322.

In an alternate embodiment, ventilation is performed through the gastrointestinal (GI) space. The system setup for perfusing the GI is identical to that shown in FIG. 3 with the exception that the small intestine is resected at the duodenum and distal ileum with the mesentery left intact. The supply tube is inserted into the resected duodenum, the scavenge tube is inserted into the resected distal ileum, and microbubbles from the supply device are pumped into and through the entire length of the small bowel.

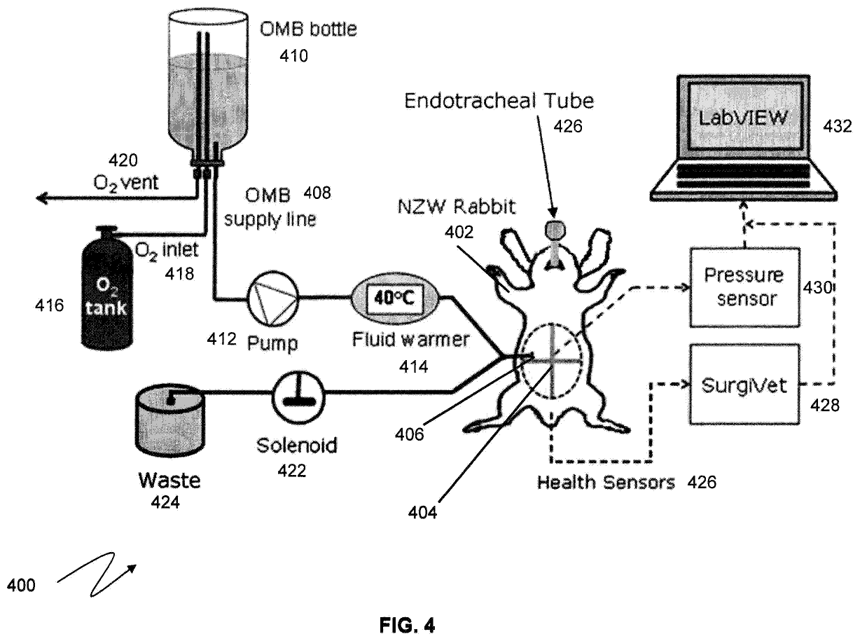

FIG. 4 illustrates another embodiment of a ventilation system 400 used in the delivery of oxygen to a subject according to the invention. In this example embodiment, a subject 402 is connected to the system 400 through its pleural cavity 404 with an inlet/outlet tube 406. The inlet/outlet tube 406 allows the subject 402 to receive oxygen microbubbles from a supply device 410 through a pump 412. The microbubbles are warmed using fluid warmer device 414.

The supply device 410 includes an oxygen tank 416 connected to an oxygen inlet 418 that prevents the supply device 410 from pressurizing while OMBs are pumped into the subject 402. There is additionally an oxygen vent 420 to assist in maintain ambient pressure in the supply device 410. The inlet/outlet tube 406 additionally allows for an expulsion of waste from the subject 402 from the pleural cavity 404. The expulsion of waste may be controlled by a solenoid valve 422, which allows the waste to drain into a waste receptacle 424. As this procedure of OMB injection is occurring, the subject's vital data is monitored with a number of health sensors 426 that are connected to a monitoring device 428. Additionally, pressure may be monitored by a pressure sensor 430. Health sensors 426 and pressure sensor 430 may be connected to a computer device 432 that may tabulate the data into a database, analyze the data, and/or used the data to provide feedback to one or more parts of the system 400. As an example, the machine computer device 432 may be connected to the supply device 410 to monitor and/or automate the injection time or quantity of OMBs.

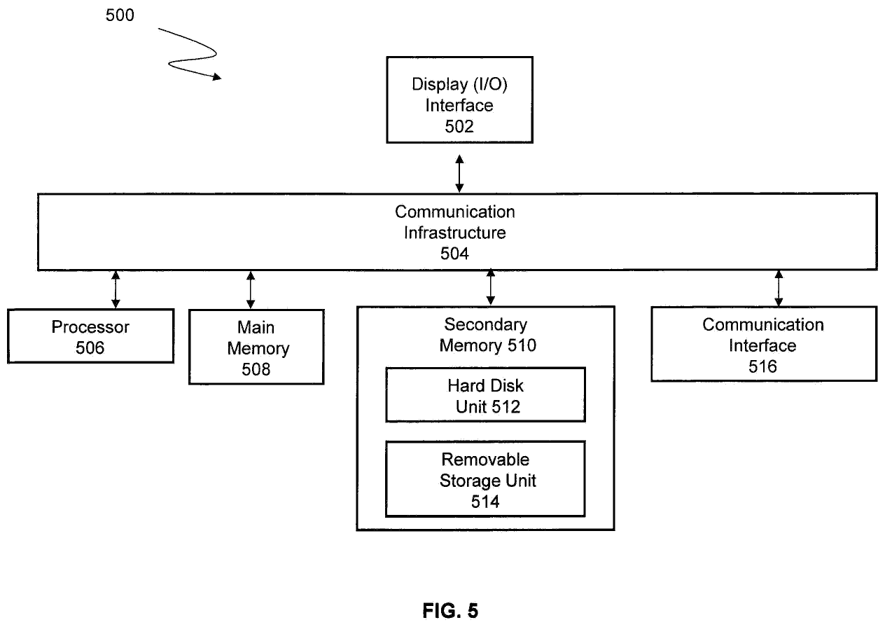

FIG. 5 illustrates an exemplary computer device 500 that may be used to implement the methods according to the invention. One or more computer systems 500 may carry out the methods presented herein as computer code.

Computer device 500 includes an input/output display interface 502 connected to communication infrastructure 504—such as a bus —that forwards data such as graphics, text, and information, from the communication infrastructure 504 or from a frame buffer (not shown) to other components of the computer device 500. The input/output display interface 502 may be, for example, a keyboard, touch screen, joystick, trackball, mouse, monitor, speaker, printer, Google Glass® unit, web camera, any other computer peripheral device, or any combination thereof, capable of entering and/or viewing data.

Computer device 500 includes one or more processors 506, which may be a special purpose or a general-purpose digital signal processor configured to process certain information. Computer device 500 also includes a main memory 508, for example random access memory (RAM), read-only memory (ROM), mass storage device, or any combination thereof. Computer device 500 may also include a secondary memory 510 such as a hard disk unit 512, a removable storage unit 514, or any combination thereof. Computer device 500 may also include a communication interface 516, for example, a modem, a network interface (such as an Ethernet card or Ethernet cable), a communication port, a PCMCIA slot and card, wired or wireless systems (such as Wi-Fi, Bluetooth, Infrared), local area networks, wide area networks, intranets, etc.

It is contemplated that the main memory 508, secondary memory 510, communication interface 516, or a combination thereof, function as a computer usable storage medium, otherwise referred to as a computer readable storage medium, to store and/or access computer software including computer instructions. Certain embodiments of a computer readable storage medium do not include any transitory signals or waves. For example, computer programs or other instructions may be loaded into the computer device 500 such as through a removable storage device, for example, a floppy disk, ZIP disks, magnetic tape, portable flash drive, optical disk such as a CD or DVD or Blu-ray, Micro-Electro-Mechanical Systems (MEMS), nanotechnological system. Specifically, computer software including computer instructions may be transferred from the removable storage unit 514 or hard disc unit 512 to the secondary memory 510 or through the communication infrastructure 504 to the main memory 508 of the computer device 500.

Communication interface 516 allows software, instructions and data to be transferred between the computer device 500 and external devices or external networks. Software, instructions, and/or data transferred by the communication interface 516 are typically in the form of signals that may be electronic, electromagnetic, optical or other signals capable of being sent and received by the communication interface 516. Signals may be sent and received using wire or cable, fiber optics, a phone line, a cellular phone link, a Radio Frequency (RF) link, wireless link, or other communication channels.

Computer programs, when executed, enable the computer device 500, particularly the processor 506, to implement the methods of the invention according to computer software including instructions.

The computer device 500 described herein may perform any one of, or any combination of, the steps of any of the methods presented herein. It is also contemplated that the methods according to the invention may be performed automatically, or may be invoked by some form of manual intervention.

The computer device 500 of FIG. 5 is provided only for purposes of illustration, such that the invention is not limited to this specific embodiment. It is appreciated that a person skilled in the relevant art knows how to program and implement the invention using any computer system.

The computer device 500 may be a handheld device and include any small-sized computer device including, for example, a personal digital assistant (PDA), smart hand-held computing device, cellular telephone, or a laptop or netbook computer, hand held console or MP3 player, tablet, or similar hand held computer device, such as an iPad®, iPad Touch® or iPhone®.

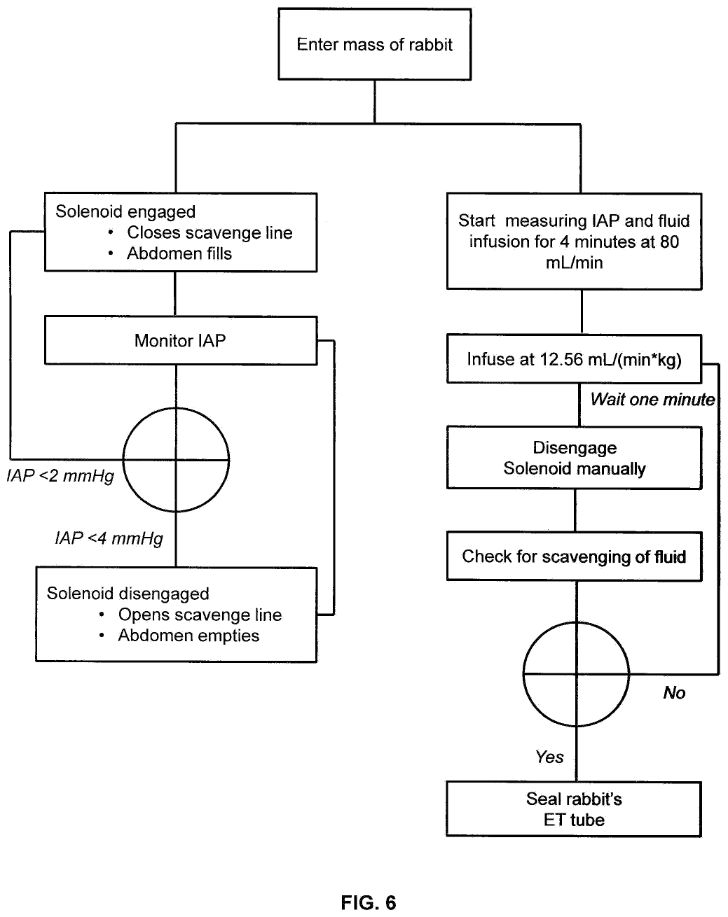

FIG. 6 is an example embodiment of the control system schematic that may be implemented at the solenoid valve to control the cavity pressure between the subject and the waste receptacle. Through monitoring the cavity pressure, the solenoid valve may be opened to relieve and lower pressure within the cavity or may be closed to increase pressure within the cavity. The opening and closing of the valve may be automated with use of the control system implemented to the computer device or may be manual with the control system providing an alarm.

### EXAMPLES

OMB production: In an example embodiment, lipids are mixed at a 9:1 molar ratio of distearoyl phosphatidylcholine (DSPC) to poly(ethylene glycol)-40 stearate (PEG40S) in saline and sonicated at low power to create the small, unilamellar liposomes. O₂ and liposomes (5 mg/mL) are then combined in the reaction chamber, where a high-power, ½-inch diameter, 20-kHz sonicator tip emulsifies the oxygen gas into micrometer-scale spheres around which phospholipid adsorbs from vesicles and micelles and self-assembles into a highly condensed (solid) monolayer coating. OMBs are separated from macroscopic foam in a subsequent flotation container and collected in syringes and centrifuged (500 g for 3 min) to form concentrated OMBs. The sonication chamber and container are jacketed with circulating coolant to maintain a constant temperature of 20° C.

OMBs are fabricated for live animal testing, in which four factors are investigated: perfusate, perfusion rate, motility drug, and method (IP or GI). OMB perfusate at 70% and 90% indicate the volume fraction of oxygen in the OMB emulsion. At 70%, the OMB emulsion's rheological properties are similar to saline and the perfusate is expected to circulate well through the IP space.

OMB size distribution measurements: OMB size distribution is varied by choosing different residence times in the flotation container (e.g., 153 min for a 10-μm diameter cut-off; 38 min for a 20-μm diameter cut-off). Size distribution is measured, for example, by electrical capacitance, light extinction/scattering, flow cytometry scatter, and optical microscopy. Alternatively, size selection may be unnecessary and may be removed from the process. OMB volume fraction is measured, for example, by gravimetric analysis and varied from 50-90 vol % by dilution with saline. Microbubble size and concentration is measured over time to investigate coalescence, Ostwald ripening and stability in storage.

OMB dissolution in oxygen-depleted media: Clinical translation of OMB technology to treat hypoxemia requires quantitative modeling of the pharmacokinetics of oxygen delivery, whether administered through the IV, IP or GI route. A model for single OMB dissolution in an oxygen-depleted medium with the presence of venous gases (30 mmHg O₂, 600 mmHg N₂ and 50 mmHg CO₂) has been developed. Results demonstrate the microbubble exhibits two dissolution regimes. First, rapid dissolution occurs as O₂ dissolves out, even as N₂ and CO₂ counter-diffuse into the bubble. Second, O₂ is depleted and slower dissolution occurs as N₂ and CO₂ dissolve out. For a 20-μm diameter microbubble, most of the oxygen is released within 10s. The bubble then carries N₂ and CO₂ (therefore acting as a blood gas scrubber for non-oxygen species).

The simulation results indicate that the highest rate of oxygen transfer for the suspension occurs with the minimal OMB residence time in the IP space. Thus, rapid mixing should provide high mass transfer coefficients for both OMB oxygen release and oxygen transfer in the fluid medium to the parietal peritoneum and visceral peritoneum membranes. The results also indicate that oxygen-release rate increases substantially for larger microbubbles. It is expected that mixing conditions (i.e., viscosity) will dominate the rate of oxygen release.

Oxygen-release rate measurements: Three trials (three measurements per trial) are taken for each OMB flow rate and size distribution. Oxygen content is plotted versus time, and the linear regime is used to determine the oxygen-release rate (ng/s). The oxygen-release rate is compared for microbubbles of two different size distributions (1-10 μm diameter vs. 10-20 μm diameter), each at three different OMB flow rates (1, 10 and 20 mL/min) and three different volume fractions (50, 70 and 90%).

IP ventilation of rat model in vivo: Using the system according to the invention, rats experienced a right pneumothorax after generally anesthetized by isoflurane (5% induction to effect) followed by sodium pentobarbital (50 mg/kg, intramuscular). Anesthesia depth is monitored and OMB perfusate is pumped into the IP space through a small incision in an upper quadrant of the abdomen. The IP incision is sutured/glued around the supply and scavenger tubing to form a seal. Intra-abdominal pressure is maintained at 8 mmHg through the use of a fluidic control solenoid valve and pressure catheter at the scavenger tube exit. Intra-abdominal pressure of 8 mmHg is used because it is typical of human insufflation pressures during laparoscopic surgery. Cardiac arrest will eventually follow pneumothorax and is caused by lack of blood oxygen (hypoxemia) and by elevated carbon dioxide levels and subsequent low blood pH (acidosis), which result from pulmonary failure. The effectiveness of IP EV is established from periodic tail-vein blood draws (5-minute intervals, total draws less than 1% body weight) and measurement of blood oxygen, carbon dioxide, pH, osmolality levels, and general blood chemistry.

Fluids used as a perfusate are saline as a control or a solution with an OMB concentration of 70% or 90% and dispensed at fluid flow rates of 0, 8, and 16 m L/min. OMB perfusate at 70% and 90% indicate the volume fraction of oxygen in the OMB emulsion. At 50%, the OMB emulsion's rheological properties are similar to saline. In certain other example embodiments, an OMB concentration between 60%-95% may be used. In certain other example embodiments, an OMB concentration of about 10%-90% may be used. At 90% volume fraction, the OMBs ability to circulate in the IP space degrades due to increased viscosity, but oxygen content is enhanced. It was calculated that perfusing OMBs at 8 mL/min provides sufficient oxygen capacity for a 400 g rat. Perfusing OMBs at 8 mL min⁻¹ provides sufficient oxygen capacity considering a 400 g rat. Perfusion at a higher rate (16 mL min⁻¹) and introducing a stagnant OMB bolus (0 mL min⁻¹). The motility enhancing drug ghrelin is administered (200 mg kg⁻¹ dose) in half of the tests via the tail vein catheter 30 minutes prior to anesthetization.

IP ventilation of rabbit model in vivo: Male New Zealand White rabbits (n=13, m=2.260±0.196 kg) were weighed and anesthetized by 5% isoflurane gas by nose cone to effect. Rabbits were then intubated with an endotracheal tube and laid in the supine position on a warming pad set at 38° C. to maintain body temperature. The abdomen of the rabbit was shaved, divided into quadrants with a marker, and sterilized. A veterinary monitor was used to monitor and record vitals via two sensors placed rectally; a pulse oximetry sensor was used to measure pulse rate and arterial oxygen hemoglobin saturation (SpO₂), and a temperature probe was used for measuring body temperature. The rabbit was given an intramuscular injection of ketamine-xylazine (35-5 mg/kg) and then gradually weaned off isoflurane and allowed to breathe room air. A small incision was made into the abdomen to allow access for placement of infusion and drainage tubing (3.2 mm inner, 4.8 mm outer diameter, Tygon) in the intraperitoneal cavity and then sutured closed. A 12G indwelling catheter and a 14G needle connected to pressure transducers were inserted into the IP cavity for measuring intra-abdominal pressure via a data acquisition and custom control system. The control system was designed to use IA pressure and a solenoid fixed to the drainage line to regulate the volume of perfusate administered into the cavity.

Perfusate was pumped through a fluid warmer set at 40.4° C. and into the IP space with a peristaltic pump at 80 mL/min for 3 minutes and then at dose of 12.6 mL/min*kg thereafter. At this time, the endotracheal tube was hermetically sealed to prevent oxygen intake and simulate complete lung failure. The fluids used as a perfusate were oxygenated saline for the control group and OMBs for the experimental group. The survival data for rabbits using saline survived 6.9±0.6 minutes. The OMB treated rabbits survived on average for 18 minutes and one outlier lived for 72 minutes after the trachea tube had been sealed; The outlier 72 minute survival time rabbit had successful circulation and IA pressure below 8 mmHg.

GI ventilation of rat model in vivo: The test setup for perfusing the GI is identical to that for perfusing the IP except that the small intestine is resected at the duodenum and distal ileum (with the mesentery left intact). The supply tube is inserted into the resected duodenum, the scavenge tube is inserted into the resected distal ileum, and perfusate is pumped into and through the entire length of the small bowel. Saline with neutral oxygen tension with respect to atmosphere is used as a third perfusate to provide a control for all experiments. Six rats are tested at the given OMB perfusion rate of 8 and 16 mL/min flow rates and the internal pressure over time was measured to find suitable conditions for infusion and scavenging. Results show that internal pressure never surpasses the upper limit of 8 mmHg and the system perfuses saline at the desired rates while maintaining physiological pressure levels in the phantom abdomen.

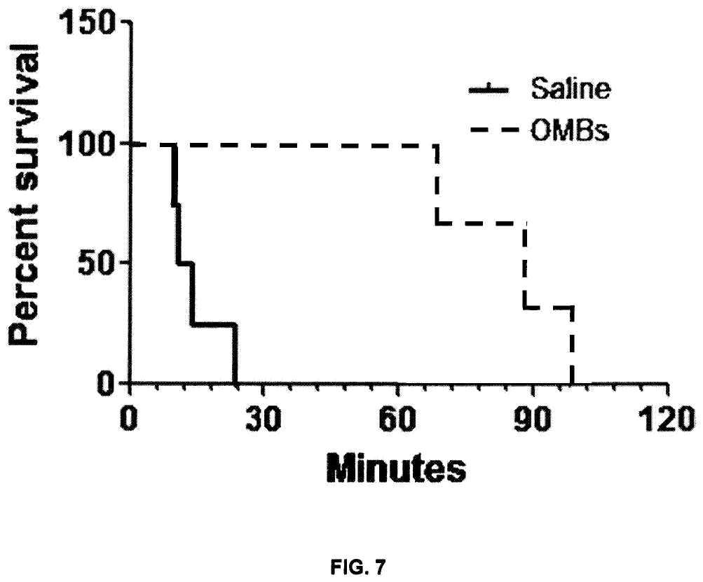

Perfusing OMBs at 8 mL min⁻¹ in 400 g rats with a right pneumothorax survival rates are promising over time. FIG. 7 illustrates a graph of survival rates over time for test subjects with a pneumothorax model and intraperitoneal injection of oxygen microbubbles compared to saline injection according to the invention. The fluids used as perfusate were (1) saline as a control or (2) a solution with an OMB concentration of ˜50 vol %. Intra-abdominal pressure was measured at the supply tube inlet to avoid high internal pressures that could damage internal organs or the IP catheter seal. Cardiac arrest eventually follows a right pneumothorax and is caused by lack of blood oxygen (hypoxemia) and elevated carbon dioxide levels and subsequent low blood pH (acidosis), which result from pulmonary failure. As shown in FIG. 7, saline control specimens survived for an average of ˜14.1 minutes after right pneumothorax in the presence of saline. In comparison, the specimen injected with OMBs survived for an average of ˜83.3 minutes after right pneumothorax, which represents a ˜8-fold improvement in survivability in comparison to the controls (p<0.5; Gehan-Breslow-Wilcoxon Test). The effectiveness of IP ventilation was established by measuring saturated arterial oxygen fraction (SAO2). Results demonstrate that cardiac arrest in hypoxemic rodents is significantly delayed by intraperitoneal administration of OMBs.

Pleural cavity ventilation of rabbit model in vivo: Male New Zealand White rabbits were weighed and anesthetized by 5% isoflurane gas by nose cone to effect. Rabbits were then intubated with an endotracheal tube and laid in the supine position on a warming pad set at 38° C. to maintain body temperature. The abdomen of the rabbit was shaved, divided into quadrants with a marker, and sterilized. A veterinary monitor was used to monitor and record vitals via two sensors placed rectally; a pulse oximetry sensor was used to measure pulse rate and arterial oxygen hemoglobin saturation (SpO₂), and a temperature probe was used for measuring body temperature. The rabbit was given an intramuscular injection of ketamine-xylazine (35-5 mg/kg) and then gradually weaned off isoflurane and allowed to breathe room air. A small incision was made into the abdomen to allow access for placement of infusion and drainage tubing (3.2 mm inner, 4.8 mm outer diameter, Tygon) in the pleural cavity and then sutured closed. A 12G indwelling catheter and a 14G needle connected to pressure transducers were inserted into the pleural cavity for measuring pressure via a data acquisition and custom control system as shown in FIG. 6. The control system was designed to use IA pressure and a solenoid fixed to the drainage line to regulate the volume of perfusate administered into the cavity.

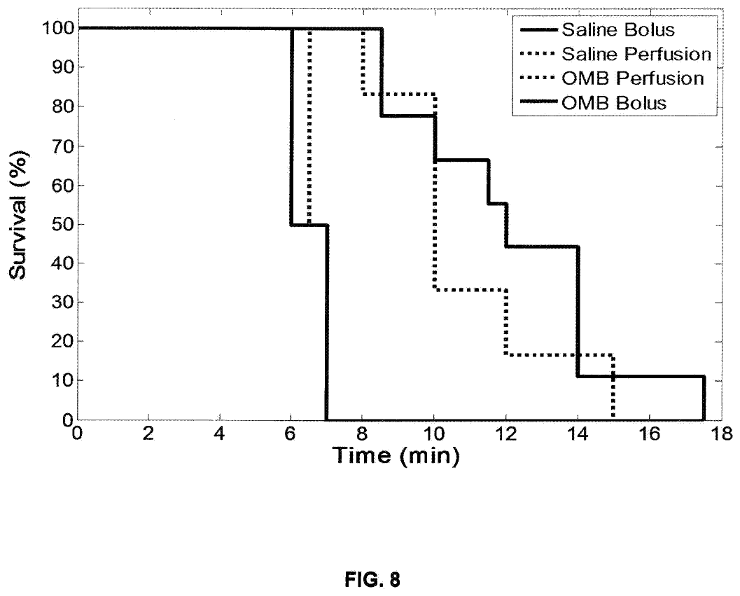

Perfusate was pumped through a fluid warmer set at 40.4° C. and into the pleural space. At this time, the endotracheal tube was hermetically sealed to prevent oxygen intake and simulate complete lung failure. The fluids used as a perfusate were oxygenated saline for the control group and OMBs for the experimental group. The survival data for rabbits using saline under a saline bolus or saline perfusion injection survived an average of seven minutes. The OMB perfusion treated rabbits survived on average for 15 minutes and OMB bolus treated rabbits survived on average for 17.5 minutes as shown in FIG. 8.

### GAS REGENERATION AND/OR SCAVENGING

It is well understood that carbon dioxide is given off as a by-product of cell metabolism and is carried by the blood through the venous system (veins) to the lungs, where it is transported through the alveoli and exhaled. The concentration of CO₂ in each breath is approximately 3.8% of the exhaled volume, and the “average” person produces approximately two pounds of carbon dioxide each day. Obviously, more CO₂ is given off by strenuous activity, while less CO₂ is produced during sedentary periods and/or during sleep.

Hypercapnia, hypercarbia, or hypercapnea is the physiological term for the condition of, and the body's response to, excessive carbon dioxide. When CO₂ is breathed into the lungs, it dissolves in the water there, diffuses across the alveolar-capillary membrane, and enters the bloodstream. As it combines with water, CO₂ forms carbonic acid, making the blood acidic—thus lowering the blood pH. When CO₂ levels become excessive, a condition known as acidosis can occur. This condition is defined as the pH of the blood becoming less than 7.35. Normally, the body maintains the balance mainly by using bicarbonate ions in the blood. As the body responds to neutralize this condition, an electrolyte imbalance—an increase of plasma chloride, potassium, calcium and sodium, can occur.

In the blood stream, CO₂ concentration is also controlled by reversible reactions with two major blood components, plasma proteins and hemoglobin. In addition, the body can use other specific mechanisms to compensate for the excess carbon dioxide. For example, breathing rate and breathing volume can increase, the blood pressure can increase, and/or the heart rate can increase, and kidney bicarbonate production (in order to buffer the effects of blood acidosis), can occur. Blood vessels in the extremities will typically constrict, restricting blood flow to these body parts. At the same time, arteries in the brain, spinal cord, and heart may dilate, so that more blood flow is diverted to maintain the function of these critical organs.

When there is exposure to very high levels of CO₂ (typically in excess of 5% or 50,000 ppm), the body's compensatory mechanisms can become overwhelmed, and the central nervous system (brain and spinal cord) functions become depressed, then ultimately fail (with death soon following)—even in the presence of sufficient oxygen to maintain normal metabolism.

While the provision of oxygen and/or other compounds using microbubbles can significantly prolong the life of an individual with compromised lung function, the removal of carbon dioxide and/or other waste materials is similarly important to the maintenance of adequate life function. For example, carbon dioxide absorbers incorporated into anesthesia machines make rebreathing possible, thus conserving gases and volatile agents, decreasing OR pollution, and avoiding hazards of carbon dioxide rebreathing. Similar absorption systems can be used for “rebreathing” devices, such as for deep sea diving. Common agents recognized for chemical absorption and binding of CO₂ include the following: chitosan; soda lime; calcium hydroxide lime; calcium hydroxide; sodium hydroxide; sodium hydroxide-coated silica; potassium hydroxide; lithium hydroxide; cuprous chloride; amine based solvents including methylamine, triethanolamine, hexamethylenetetramine, cyclohexylamine, pyridine, diphenylamine, ethylenediamine, ethylpiperidine, benzimdazole, triazine, triphenylphosphine, and dimethylphosphine. Soda lime includes an activator such as NaOH or KOH. Silica and kieselguhr may be added as hardeners.

Some carbon dioxide absorbent compounds are associated with potential complications, such as: (1) Compound A can be produced in soda lime (lethal at 130-340 ppm, or renal injury at 25-50 ppm in rats; but incidence of toxic [hepatic or renal] or lethal effects in millions of humans are comparable to desflurane). (2) Carbon monoxide is produced by (desflurane::: enflurane >isoflurane) >>(halothane=sevoflurane) and is a greater issue in dry absorbent.

The strong bases (NaOH, KOH which function as activators) have been convincingly implicated in the carbon monoxide problem with the ethyl-methyl ethers, and the generation of Compound A by sevoflurane. Two more definitive approaches to dealing with these problems have surfaced: (1.) Lithium-containing (Litholyme) or lithium-based (Spiralith) absorbents; and (2.) Absorbents which lack activators—Lithium hydroxide lime is an effective carbon dioxide absorbent, and is free of the strong bases (NaOH, KOH). LiCl acts as the catalyst to accelerate the formation of CaCO3; ethyl violet acts as the indicator; and it does not contain KOH or NaOH. LiCl: (1) does not produce Compound A, even when the absorbent is fully desiccated, (2) when exhausted, undergoes a permanent color change which will not revert upon resting the absorbent, (3) generates less heat than soda lime, and (4) is comparable in price to soda lime.

In various embodiments, color change agents responsive to CO₂ presence may optionally be utilized to indicate when the selected compound has reached CO₂ saturation and will desirably be replaced for proper management of anesthesia gases in subsequent patient use. Indicators for soda lime (such as ethyl violet) can be colorless when fresh, and purple when exhausted, because of pH changes in the granules.

In various embodiments, the invention includes the incorporation of devices and/or methods for removing CO₂ of other waste products from the bloodstream of a treated patient in a minimally invasive manner, which is highly effective in cases of lung injury and compromised ability to pass CO₂ via the lung, and which does not require vascular access for blood gas alteration.

In various embodiments, lipid oxygen microbubbles (OMB) can be utilized to significantly decrease the concentration of CO₂ in blood when delivered via the rectum to the colon, via surgical openings to the peritoneum and/or other body cavities. OMBs are designed for high oxygen carrying capacity and rapid oxygen delivery to the peritoneum, and similar or other types of microbubbles (including other gases and/or gas mixtures) can similarly or simultaneously be utilized for CO₂ removal. A 70% suspension of OMBs at 1.0 bar and 37° C. contains an estimated 0.88 mg-O₂ mL^{.1}, compared to 0.31, 0.62 and 0.57 mg-O₂ mL^{.1} for blood, liposomal hemoglobin and pure PFC, respectively. However, if oxygen-carrying capacity were the only criterion for successful colon or peritoneal oxygenation, then it should be expected that ventilation with pure oxygen (1.26 mg-O₂ mL^{.1}) should provide clinically relevant oxygenation—which is not the case. Applicant has discovered that the limited uptake in these prior direct peritoneal-ventilator studies can be attributed to various problems stemming from the injection of a single bolus of free gas into the peritoneal cavity.

The surface area of a macroscopic ventilator bubble is minimized owing to the high surface free energy of the uncoated gas/water interface. The surface tension contracts the interface until the configuration of the ventilator bubble has a minimal surface area This limits the peritoneal surface area in contact with the ventilator bubble and therefore limits the transport area available to the peritoneum. In contrast, OMBs and/or other microbubbles are typically not under tension and may freely disperse throughout the colon or peritoneal cavity and make intimate contact with the villi of the mesothelium and other tissues, thus maximizing available surface area for transport and minimizing the thickness of the stagnant fluid film that overlays the mesothelium. OMBs and/or other microbubbles thus provide a much higher overall oxygen delivery rate and/or CO₂ removal rate to the circulating blood via the colon and peritoneum, which adequately explains why OMBs can be successful in peritoneal oxygenation where pure oxygen ventilation will fail.

Applicant has also discovered that the diffusion of gases from OMBs to surrounding tissues and the bloodstream can be a “two way street”—that is, a similar diffusion of CO₂ from areas of higher concentration in the bloodstream and/or tissues of the body occurs towards the lower CO₂ concentrations of the resident OMB within the abdominal cavity or colon. In fact, the level of blood CO₂ markedly decreases in the presence of OMBs or other microbubbles within body cavities as the CO₂ diffuses from the blood to the lipid gas emulsion, resulting in an increased concentration of CO₂ within the lipid emulsion as the oxygen gas diffuses into the blood. In various embodiments, lipid microbubbles can be preferentially formed with oxygen, nitrogen, or other gas or combination of gases intended for exchange with CO₂ or diffusion from the tissue of an organism to the lipid microbubble.

Because of increasing levels of CO₂ in the OMBs would eventually reduce the amount of CO₂ diffusion from the blood stream (i.e. due to the decreasing concentration differential or gradient), various embodiments of Applicant's invention include the use of a CO₂ absorbent or “scrubber” within the body cavity containing the OMBs, which desirably removes CO₂ from the OMB's and potentially “refreshes” their ability to transfer additional CO₂ from the bloodstream. In many cases, the useful life of the OMB's may be greatly extended by such refreshing, and there may be little or no need for additional OMB's to be introduced to a given patient.

The invention discloses a catheter designed for trans-rectal delivery to the rectum or otherwise advanced to the colon of a patient (or other body cavity, including the peritoneal cavity) for the purpose of regulating CO₂ levels in the circulating blood. The assembly of the invention will desirably include a length of catheter body extending externally to the patient into and/or across the rectum to the colon. The distal portion of the catheter assembly includes an absorbing region for the chemical bonding of CO₂ with a catheter delivered substrate. The CO₂ absorbing material positioned at a distal portion of the catheter can consist of the eligible materials discussed above as having high affinity for chemical bonding of CO₂. The catheter may reside within the colon or abdominal cavity for a sufficient period of time to allow CO₂ diffusion to result in chemical bonding of the CO₂ to the substrate material contained within the catheter assembly. In other embodiment, the catheter assembly may be modified for placement to the abdominal cavity for gas transfer across the peritoneum.

In various embodiments, the catheter may incorporate a circulatory or regeneration system that allows the catheter to remain within the body cavity, or in other embodiments the catheter may be removed and/or replaced after a sufficient indwelling period to allow for CO₂ absorption and bonding to the substrate. In many embodiments, CO₂ is desirably effectively removed first from the bloodstream and then from the body of the treated patient. The beneficial gas transfer properties of gas containing lipid microbubbles will desirably promote the rapid diffusion of CO₂ from the bloodstream across the microbubble reservoir within the colon or abdominal cavity to the substrate contained within the catheter assembly. On full saturation of CO₂ within the substrate, a color change of the substrate may indicate the need for replacement to achieve further CO₂ bonding for removal of CO₂ from the patient. The catheter assembly may remain indwelling for extended periods in combination with ventilator therapy, or be placed for a discrete event in critical care, or placed to aid blood gas management during patient transport or intubation procedures. Color change may provide visual confirmation to attending health care personnel that CO₂ has been effectively removed from the bloodstream via the microbubble medium. Indwelling time and blood gas measurements at periodic intervals may indicate the absorption status of the absorption region of the catheter.

In an acute care setting, medication and fluid administration is generally limited to oral, intravenous, or intraosseous routes, but in many patients, particularly in the emergency or critical care settings, these routes are often unavailable or time-consuming to access. Devices are currently available that offer an easy route for administration of medications or fluids via rectal mucosal absorption (also referred to as proctoclysis in the case of fluid administration and subsequent absorption), which can similarly be utilized for placement and/or regeneration of OMBs or other microbubbles such as described herein. Although originally intended for the palliative care market, the utility of these devices in the emergency setting has been described. Reports of patients being treated for dehydration, alcohol withdrawal, vomiting, fever, myocardial infarction, hyperthyroidism, and cardiac arrest have shown success with administration of a wide variety of medications or fluids (including water, aspirin, lorazepam, ondansetron, acetaminophen, methimazole, and buspirone). Device placement is straightforward, and based on the observation of expected effects from the medication administrations, absorption is rapid.

In addition to gas transfer, OMB's or other microbubbles can also represent a significant potential improvement in the available routes of administration used for medications and fluids and the like.

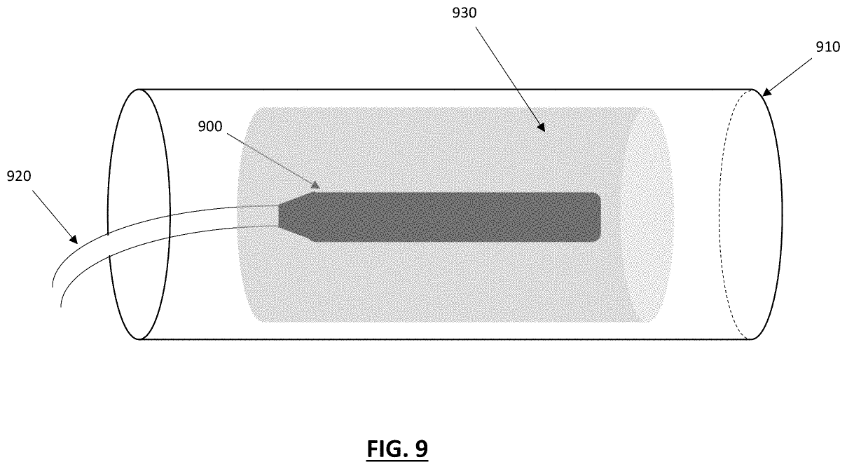

FIG. 9 illustrates one exemplary embodiment of a preferred device and placement of the invention. The drawing illustrates a discrete length of the colon 910. The colon 910 has been filled by infusion of a lipid shell gas filled microbubble foam 930. In a preferred embodiment the gas contained in the lipid microbubble can be oxygen. A catheter assembly 900 can be inserted into the colon through an existing opening (and/or via an artificial incision) with a retention tape or tube 920 placed extending from outside the patient across the rectum (not shown) such that the distal end resides within the colon. Desirably, in some embodiment the catheter can be inserted in a lower profile configuration and enlarged within the body cavity in a known manner. Within the catheter assembly a CO₂ absorbing medium can be contained, and in some embodiments the walls of the catheter assembly may be perforated and/or may incorporate a gas permeable material. The CO₂ absorbing medium will desirably come into contact with the lipid microbubble in a manner which allows gas diffusion from the microbubble to the medium contained within the catheter. In FIG. 9, gas exchange can be achieved across a gas permeable membrane which contains the filler medium. Microbubble contact with the medium may be by means of perforations in the structure containing the medium, such that there is direct contact of the medium to the microbubble agent. In some embodiments, optional circulation of the microbubbles (i.e., using a rotating paddle or other mixing device within the body cavity) may be accomplished to aid in gas flow and/or diffusion.

In FIG. 9, the catheter can be delivered in a collapsed or low diameter configuration to the colon, desirably in contact with lipid microbubble resident in the colon. The lipid microbubble agent may be delivered prior to catheter placement, delivered during catheter placement, delivered following catheter placement, or removed and exchanged while the catheter device is in place and remains indwelling. The medium for CO₂ absorption may be present at the time of delivery, or may be delivered to a distal catheter region after device placement. The distal catheter region may expand in diameter after placement to the colon to allow for increased volume of the absorbing medium to enter into the distal region of the catheter. The catheter and absorbing medium may remain resident to allow gas diffusion across the lipid microbubble, such that gas in the lipid microbubble diffuses to and enters the bloodstream, and CO₂ in the bloodstream can diffuse to the lipid microbubble and medium, where chemical reaction will desirably bond the CO₂ within the medium. On removal from the colon and inspection, a color change of the medium material may indicate the amount of CO₂ absorbed to the medium, to determine capacity for further use and CO₂ absorption for removal. Such color change or other condition of the absorbing medium may be observed remotely, if desired. In some embodiments, the medium may optionally be removed independent of the catheter, thereby allowing the catheter to remain in place while the medium is exchanged for additional CO₂ storage. The catheter and medium may be removed and exchanged or replaced as a single unit.

In various embodiments, the catheter may include an opening, a tube, a tubular circuit or other system for delivering, removing and/or recirculating the microbubbles after catheter placement within the body cavity. In other embodiments, an internal and/or external pump or other circulating device may be incorporated in or attached to the catheter body to desirably circulate the microbubbles within the body cavity, if desired.

Many current medical techniques for systemic CO₂ removal typically rely on access to the bloodstream and CO₂ removal by filtration techniques. Extracorporeal carbon dioxide removal (ECCO₂R) provides an alternative or supplement to mechanical ventilation by removing carbon dioxide directly from the blood using techniques similar to kidney dialysis. The procedure can be performed as part of comprehensive treatment for acute respiratory failure in the intensive care unit. Many patients with respiratory failure require the assistance of a ventilator to provide life sustaining oxygenation and carbon dioxide removal. Unfortunately, the injured lung is often susceptible to additional damage by the positive pressure exerted by the ventilator, leading to additional injury, complications, and increased mortality. Reducing ventilator pressures has been one of the most important interventions shown to improve outcomes in these critically ill patients. Use of these lung-protective ventilation strategies in which the ventilator is “turned down” can lead to a critical accumulation of carbon dioxide in the blood. Extracorporeal CO₂ removal can allow carbon dioxide to be removed from the blood independently of the failing lungs with an extracorporeal device in a manner similar to kidney dialysis, thus enabling implementation of safer mechanical ventilation settings

The proposed invention allows for the removal of CO₂ from the bloodstream and from the body by methods which will not require access to the bloodstream, thus greatly reducing and/or avoiding risk of vascular access complications, thrombus formation, and bleeding complications typically associated with percutaneous access techniques.

While various embodiments of the disclosure discuss the benefits of carbon dioxide removal using oxygen microbubbles (OMBs), it should be understood that various other gases (including pure gases, gas combinations, and/or atmospheric gas or various combinations thereof in varying concentrations and/or ratios) may be utilized in place of oxygen, or in combination with oxygen, as a carbon dioxide removal medium, and the gas within the microbubbles need not necessarily be oxygen in the various embodiments disclosed herein. While an oxygen filled microbubble may be particularly useful in treatments for providing both oxygen delivery and carbon dioxide removal, the lipid oxygen microbubbles or OMB's described herein in any of the disclosed embodiments may be utilized with a range of alternative gases and/or gas combinations to accomplish the carbon dioxide and/or waste removal functions described herein.

While the disclosure is susceptible to various modifications and alternative forms, specific exemplary embodiments of the invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

### HEADINGS

The headings provided herein are merely for the reader's convenience and should not be construed as limiting the scope of the various disclosures or sections thereunder, nor should they preclude the application of such disclosures to various other embodiments or sections described herein.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

What is claimed is:

1. A method of removing a gas from a circulatory system of a subject, comprising the steps of:
delivering a plurality of microbubbles containing a first gas into an existing gastrointestinal body cavity of the subject, the first gas having a first percentage by volume;
inserting a gas collecting device into the first plurality of microbubbles located within the gastrointestinal body cavity, the gas collecting device comprising a gas permeable outer layer and a material capable of absorbing the selected gas;
wherein a gas located within the circulatory system of the subject diffuses through a tissue wall of the gastrointestinal body, through the plurality of microbubbles and through the gas permeable outer layer and is absorbed by the gas absorbing material.

2. The method of claim 1, where the gas collecting device contains a gas absorbing material selected from one of the members of a group consisting of chitosan; soda lime; calcium hydroxide lime; calcium hydroxide; sodium hydroxide; sodium hydroxide-coated silica; potassium hydroxide; lithium hydroxide; cuprous chloride; amine based solvents, methylamine, triethanolamine, hexamethylenetetramine, cyclohexylamine, pyridine, diphenylamine, ethylenediamine, ethylpiperidine, benzimdazole, triazine, triphenylphosphine, dimethylphosphine and any combinations thereof.

3. The method of claim 1, wherein the plurality of microbubbles comprise microbubbles 10 micrometers or smaller in diameter.

4. The method of claim 1, wherein the gas collecting device is inserted into the existing gastrointestinal body cavity of the subject via a naturally existing opening.

5. A method of removing carbon dioxide from a circulatory system of a subject, comprising the steps of:
delivering a plurality of microbubbles containing a first gas into a body cavity of the subject; and
inserting a carbon dioxide collecting device into the first plurality of microbubbles located within the body cavity, the carbon dioxide collecting device comprising a gas permeable outer layer and a material capable of absorbing carbon dioxide gas;
wherein a carbon dioxide gas located within the circulatory system of the subject diffuses through a tissue wall of the body cavity, through the plurality of microbubbles and through the gas permeable outer layer and is absorbed by the carbon dioxide absorbing material.

6. The method of claim 5, wherein the body cavity of the subject is a colon of the subject and the carbon dioxide collecting device is inserted into the colon via a rectum of the subject.

7. The method of claim 5, wherein the body cavity of the subject is a peritoneal cavity of the subject and the carbon dioxide collecting device is inserted into the peritoneal cavity via a surgically created opening.

8. The method of claim 5, wherein the plurality of microbubbles comprise microbubbles 10 micrometers or smaller in diameter.

9. The method of claim 5, wherein the carbon dioxide collecting device is inserted into the body cavity via a naturally existing opening.

10. The method of claim 5, wherein the carbon dioxide collecting device is removable from the body cavity independent of the plurality of microbubbles.

11. The method of claim 5, wherein the plurality of microbubbles is removable from the body cavity independent of the carbon dioxide collecting device.

12. The method of claim 5, wherein the carbon dioxide collecting device may be inserted into the body cavity under radiologic guidance.

13. The method of claim 5, wherein the carbon dioxide collecting device may be inserted into the body cavity over a guidewire under radiologic guidance.

14. A method of removing carbon dioxide from a circulatory system of a subject, comprising the steps of:
delivering a plurality of microbubbles containing a first gas into an existing peritoneal body cavity of the subject; and
inserting a carbon dioxide collecting device into the peritoneal body cavity, the carbon dioxide collecting device comprising a gas permeable outer layer and a material capable of absorbing carbon dioxide gas;
wherein a carbon dioxide gas located within the circulatory system of the subject diffuses through a tissue wall of the peritoneal body, through the plurality of microbubbles and through the gas permeable outer layer and is absorbed by the carbon dioxide absorbing material.

15. The method of claim 14, wherein the carbon dioxide collecting device is inserted into the existing gastrointestinal body cavity of the subject via a surgically created opening.

16. The method of claim 14, wherein the plurality of microbubbles comprises microbubbles from 10 to 25 micrometers in diameter.

17. The method of claim 14, wherein the plurality of microbubbles comprises microbubbles from 25 to 100 micrometers in diameter.

18. The method of claim 14 wherein the plurality of microbubbles comprises microbubbles from 100 to 500 micrometers in diameter.

19. The method of claim 14, wherein the plurality of microbubbles are delivered into the existing peritoneal body cavity through an opening in the carbon dioxide collecting device.

20. The method of claim 14, wherein the plurality of microbubbles are delivered into the existing peritoneal body cavity through a tube of the carbon dioxide collecting device.
